(19) United States
(12) Patent Application Publication
(10) Pub. No.: US 2026/0047953 A1
(43) Pub. Date: Feb. 19, 2026

(54) EJACULATION CONTROL NERVE REGULATING DEVICE

(71) Applicant: Guangzhou Aoxiang Medical Technology Co., Ltd., Guangzhou City (CN)

(72) Inventors: Xianglin DU, Guangzhou City (CN); Chunhua Deng, Guangzhou City (CN)

(73) Assignee: Guangzhou Aoxiang Medical Technology Co., Ltd., Guangzhou City (CN)

(21) Appl. No.: 19/296,457

(22) Filed: Aug. 11, 2025

(30) Foreign Application Priority Data
Aug. 16, 2024 (CN) 202411126478.9

(51) Int. Cl. A61F 5/41; A61N 1/04; A61N 1/36

(52) U.S. Cl. CPC A61F 5/41; A61N 1/0408; A61N 1/0472; A61N 1/36034; A61F 2005/418

(57) ABSTRACT

An ejaculation control nerve regulating device comprises a main unit and a stimulating component. The main unit includes an electrical stimulation circuit and an outputting electrode electrically connected to the electrical stimulation circuit. The electrical stimulation circuit has a multi-frequency stimulation circuit and a mixed-frequency circuit electrically connected to each other. The stimulating component has a contacting electrode and a stimulating electrode electrically connected to each other. The electrical stimulation circuit outputs the mixed-frequency stimulation current to the outputting electrode via the multi-frequency stimulation circuit and the mixed-frequency circuit, and the outputting electrode contacts the contacting electrode of the stimulating component and conducts the mixed-frequency stimulation current to the stimulating component. The electrical stimulation circuit outputs the mixed-frequency stimulation current via a weighted frequency mixing control method or a time-based frequency mixing control method.

### BACKGROUND

### 1. Field of the Invention

The present invention relates to an electronic device, especially to an ejaculation control nerve regulating device that is easy to carry and use.

### 2. Description of the Prior Arts

Premature ejaculation is one of the most common male sexual dysfunctions and the causes include mental factors, hormone imbalance, hypersensitive glans, and other comorbidities such as chronic prostatitis (CP), chronic pelvic pain syndrome (CPPS), and other neurological disorders. In some patients, an excessive number of the dorsal nerves of penis leads to overly strong sexual stimulation signals, easily reaching the excitation threshold and thus resulting in premature ejaculation. Such patients may undergo a surgery to remove some of the dorsal nerves of penis, but the effect of said surgery is unstable: insufficient removal may not improve the hypersensitivity, while excessive removal leads to the risk of loss of functions.

The existing nerve regulating solutions mainly focus on applying ultra-low frequency electrical stimulation (below 1 kHz) and ultra-high frequency electrical stimulation (above 200 kHz). The mechanism of low frequency electrical stimulation is not fully explored yet and is mainly recognized as interfering with or exciting nerves. The persistence of the interference effect is very short, disappearing immediately as long as the electrical stimulation stops, and the nerves would rapidly revert to be sensitive. Since the interference effect lasts short, some electrical stimulation solutions even require the equipment to operate during sexual intercourse, which may disturb the normal course of sexual intercourse and thus fail to meet the actual needs of the patients. Besides, treatment with the ultra-low frequency electrical stimulation needs to last 30 minutes per time and about 2 to 3 times per week, which may start being partially effective after continuously treating for 2 to 3 months, and the premature ejaculation is easy to recur once the treatment stops; the prolonged and complicated treatment largely lowers patient adherence. Moreover, improper electrical stimulation may result in enhancement of neural circuit excitation and further lead to increased nerve sensitivity. The ultra-high frequency electrical stimulation has risks of affecting general sexual functions like erection or erectile rigidity due to thermocoagulation or excessive nerve conduction blockade.

On the other hand, existing nerve regulation equipments are mostly large equipment or integrated equipment, and said large equipment is usually installed in the hospital and the patient needs to frequently go to the hospital for treatment. The integrated equipment may be for home use, but the integrated equipment cannot sleeve on the penis for a long time due to the volume and weight of the integrated equipment; in particular, the penis is a unique body part, and it's generally challenging to maintain an erection for a prolonged period, and thus a prolonged continuous stimulation cannot be conducted by applying the integrated equipment to the penis. Furthermore, due to the dispersed nerve distribution on the penis, a traditional and general single-point electrode May provide a very limited nerve coverage when applied directly to the penis, which directly impacts the therapeutic effect.

### SUMMARY

To overcome the shortcomings, the present invention provides an ejaculation control nerve regulating device to mitigate or obviate the aforementioned problems.

The main objective of the present invention is to provide an ejaculation control nerve regulating device that is convenient for carrying and using, and is safe and effective.

The ejaculation control nerve regulating device comprises a main unit and a stimulating component. The main unit has an electrical stimulation circuit and an outputting electrode. The electrical stimulation circuit includes a multi-frequency stimulation circuit and a mixed-frequency circuit. The multi-frequency stimulation circuit is capable of generating electrical stimulation currents in various frequencies, and the mixed-frequency circuit controls the electrical stimulation circuit to output a mixed-frequency stimulation current. The outputting electrode is mounted on and exposed on an external surface of the main unit and is electrically connected to the electrical stimulation circuit, receiving the mixed-frequency stimulation current output from the electrical stimulation circuit. When in use, the outputting electrode of the main unit contacts the contacting electrode of the stimulating component, thereby conducting the electrical stimulation current and/or the mixed-frequency stimulation current generated by the electrical stimulation circuit to the stimulating electrode.

The benefits of the ejaculation control nerve regulating device in this disclosure are:
(1). The ejaculation control nerve regulating device in this disclosure is small and is provided with split-unit design, thus being easy to carry and meanwhile preventing the penis from experiencing great pressure during long-term treatment as treated with the integrated nerve regulating device.
(2). The main unit and the stimulating component of the ejaculation control nerve regulating device in this disclosure conduct the electrical stimulation by contacting, avoiding usage of wires, thus being convenient for storage and carrying, and preventing entangling of wires and cables of the power source when in use.
(3). The stimulating component of the ejaculation control nerve regulating device in this disclosure is made of an elastic material and is size-adjustable according to the penis of the patient, and the stimulating electrode being curved is capable of covering all of the nerves of the penis, ensuring the thoroughness and the accuracy of the treatment; the design of gap distance to the stimulating electrode is capable of reaching the effective electric field range, and thus is capable of effectively influencing the nerves.
(4). The ejaculation control nerve regulating device in this disclosure outputs the mixed-frequency stimulation current to treat premature ejaculation from multiple stimulation currents having frequency ranging from 10 kHz to 200 kHz via the time-based frequency-mixing control method or the weighted frequency-mixing control method; with the mechanisms of enhancing the brain center controlling over ejaculation and lowering excitation of the dorsal nerves of penis, not only increases the effective rate of treating premature ejaculation with nerve regulation, but also reduces the risks to the patient of experiencing adverse reactions like erectile dysfunction and erectile rigidity decreasing, increasing safety.
(5). The ejaculation control nerve regulating device in this disclosure may treat electrical stimulation via the mixed-frequency stimulation current, and thus reduces treatment time, generally treating 30 to 60 seconds per time and being effective immediately after treatment.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a main unit of an ejaculation control nerve regulating device in accordance with the present invention;

FIG. 2 is a perspective view of a stimulating component of the ejaculation control nerve regulating device in accordance with the present invention;

FIG. 3 is an exploded view of the main unit of the ejaculation control nerve regulating device in FIG. 1;

FIG. 4 is a perspective view of the ejaculation control nerve regulating device in accordance with the present invention, demonstrating the usage;

FIG. 5 is a block diagram of a weighted frequency mixing control method of the ejaculation control nerve regulating device in accordance with the present invention;

FIG. 6 is a block diagram of a time-based frequency mixing control method of the ejaculation control nerve regulating device in accordance with the present invention;

FIG. 7 is a front view of a stimulating component of Embodiment A of the ejaculation control nerve regulating device in accordance with the present invention;

FIG. 8 is a back view of the stimulating component of Embodiment A of the ejaculation control nerve regulating device in accordance with the present invention; and

FIG. 9 is a block diagram showing connection between an electrical stimulation circuit and an outputting electrode of the ejaculation control nerve regulating device in accordance with the present invention.

### DETAILED DESCRIPTION

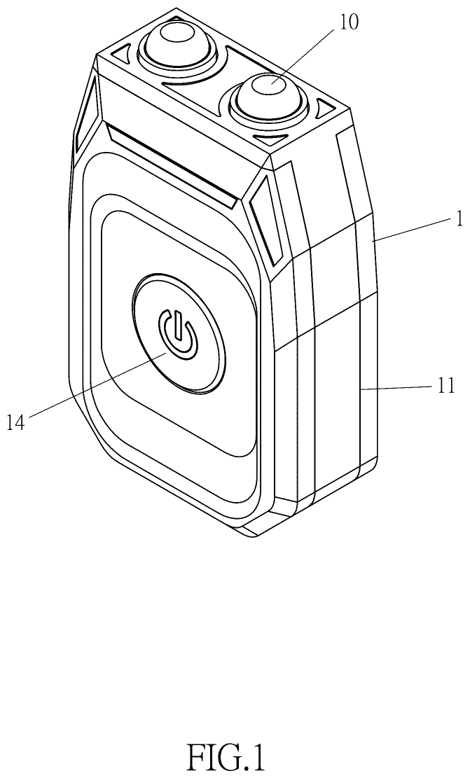

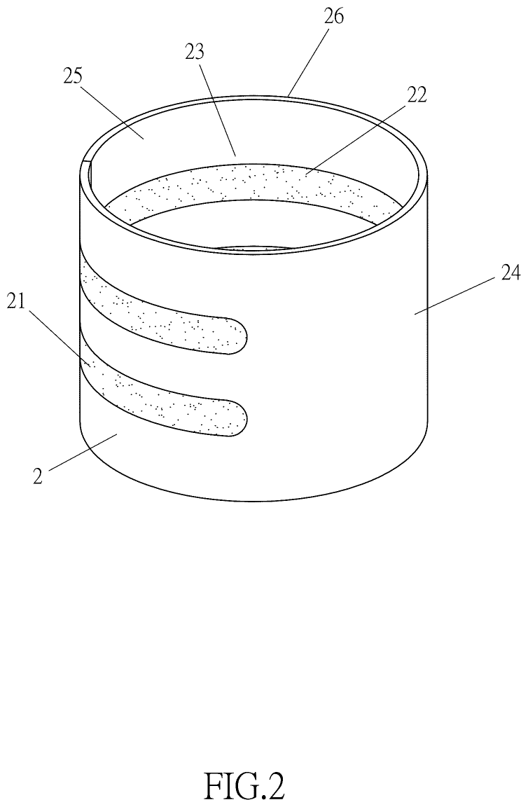

With reference to FIGS. 1 and 2, an ejaculation control nerve regulating device in accordance with the present invention comprises a main unit 1 and a stimulating component 2. The main unit 1 includes an outputting electrode 10, and the outputting electrode 10 is mounted and exposed on a surface of a casing 11 of the main unit 1. By the outputting electrode 10 contacting the stimulating component 2, a mixed-frequency stimulation current generated by the main unit 1 is conducted to the stimulating component 2.

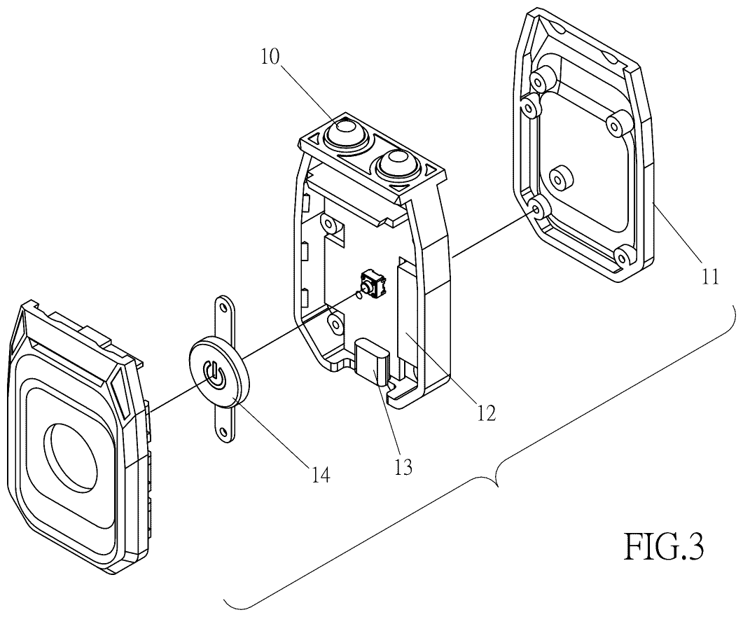

Further with reference to FIG. 3, an electrical stimulation circuit 12 and a power source 13 electrically connected to the electrical stimulation circuit 12 are mounted in the casing 11 of the main unit 1, and the power source 13 supplies power to the electrical stimulation circuit 12. A switch 14 is mounted on the external surface of the casing 11.

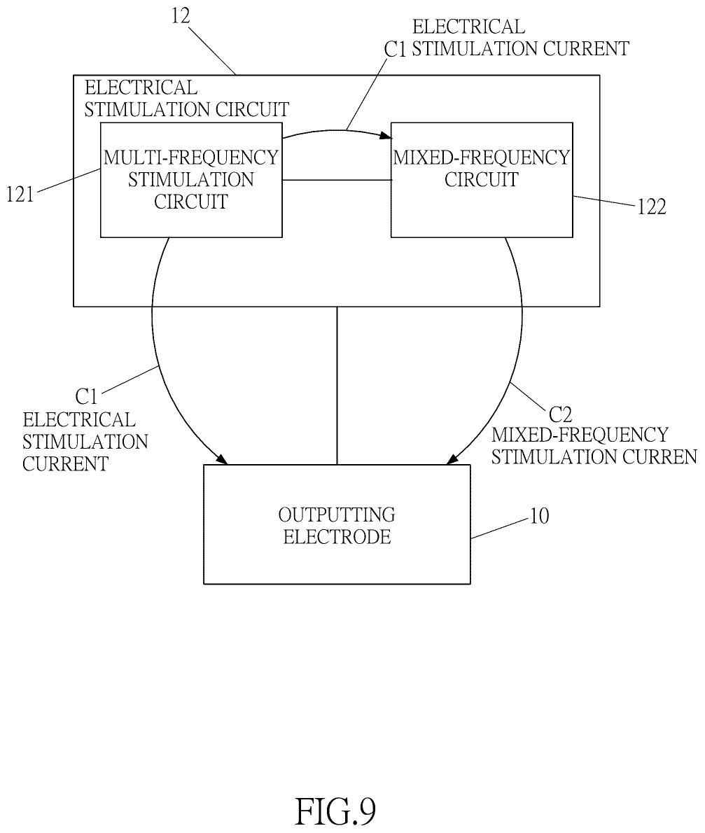

Further with reference to FIG. 9 together, the electrical stimulation circuit 12 includes a multi-frequency stimulation circuit 121 and a mixed-frequency circuit 122; the multi-frequency stimulation circuit 121 is capable of generating electrical stimulation currents C1 in various frequencies, the mixed-frequency circuit 122 controls the electrical stimulation circuit 12 to mix the electrical stimulation currents C1 with various frequencies and output a mixed-frequency stimulation current C2. The electrical stimulation currents C1 in various frequencies may include low-frequency-stimulation currents and high-frequency-stimulation currents.

Preferably, the multi-frequency stimulation circuit 121 is at least capable of outputting a low-frequency stimulation current and a high-frequency stimulation current. A frequency range of the low-frequency stimulation current is between 10 kHz and 100 kHz, and a frequency range of the high-frequency stimulation current is between 100 kHz and 200 kHz. The low-frequency stimulation current is weak in penetrating cell membranes, but has good capability of inducing action potential of nerves, enhancing the connection between nerves and brain and the feedback feeling within the treatment, and thus enhances the brain center controlling over ejaculation. The high-frequency stimulation current is capable of penetrating cell membranes, and the impedance rapidly decreases as the frequency increases, and the capacitive component increases; a significant portion of the current then begins to flow into the nerve cells, enabling it to affect intracellular activities of the nerve cells and interfere with the communication among the nerve cells, thereby raising the ejaculation threshold; in addition, the low-frequency stimulation current and the high-frequency stimulation current are applied to the dorsal nerves of penis and thus obtain a better safety, and the nerves are trained for having an inhibitory effect without damaging or blockade in nerve conduction, thereby preventing the patient from loss of normal sexual functions like erection and erectile rigidity.

When the frequency of the stimulation current is below 10 kHz, the current primarily flows within extracellular fluid, and thus is only capable of inducing the action potential over cell membranes, so the patient would get a brief sensory stimulation or muscle twitching, which is hard to produce inhibitory effect in neural excitation; when the frequency of the stimulation current is higher than 200 kHz, the capacitive influence decreases, and the impedance is primarily determined by intracellular fluid and changes little, thereby easy to produce thermocoagulation or nerve conduction blockade, resulting in much severe damage to the patient.

As shown in FIG. 2, the stimulating component 2 has a contacting electrode 21 and a stimulating electrode 22; the stimulating component 2 forms an accommodating space 23 in an inner side for enveloping a stimulated site; the contacting electrode 21 is mounted on an outer surface 24 of the stimulating component 2, and the stimulating electrode 22 is mounted on an inner surface 25 of the stimulating component 2, and the contacting electrode 21 and the stimulating electrode 22 electrically connect with each other; an opening 26 is disposed on at least an end of the accommodating space 23.

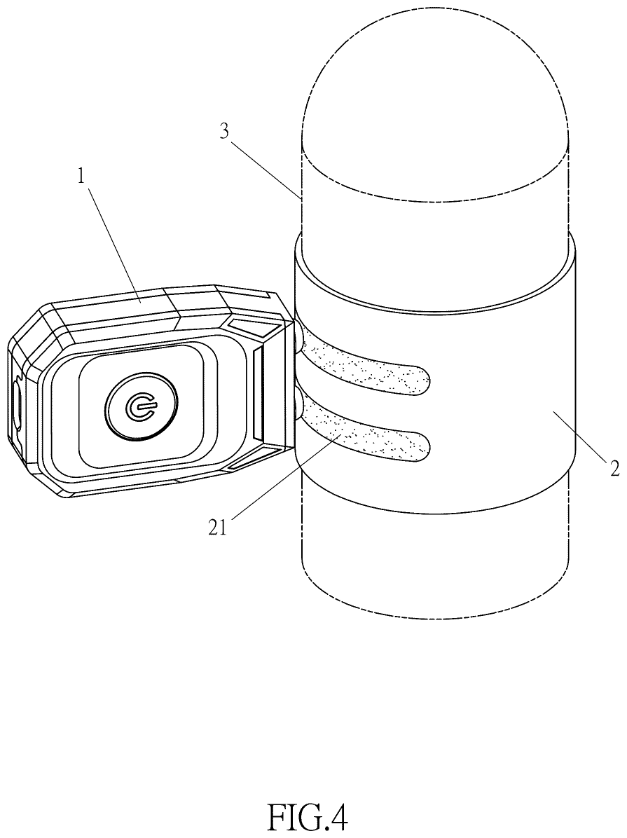

As shown in FIG. 4, when in use, the outputting electrode 10 needs to contact the contacting electrode 21 of the stimulating component 2, for conducting the mixed-frequency stimulation current output from the main unit 1 to the stimulating electrode 22 via the outputting electrode 10 and the contacting electrode 21.

In addition, a control method configured to control an electrical stimulation circuit of an ejaculation control nerve regulating device in the present invention comprises:
Outputting multiple stimulation currents: turn on a power source and power up an electrical stimulation circuit, and a multi-frequency stimulation circuit of the electrical stimulation circuit generates and outputs multiple electrical stimulation currents to a mixed-frequency circuit, and a frequency of at least one of the electrical stimulation currents ranges from 100 kHz to 200 kHz;
Frequency-mixing and outputting: the mixed-frequency circuit receives the multiple electrical stimulation currents and outputs a mixed-frequency stimulation current under a mixed-frequency mode to an outputting electrode.

Further, the control method as mentioned above, wherein the mixed-frequency mode is that the mixed-frequency circuit simultaneously receives the multiple electrical stimulation currents, and mixes electrical energies of the multiple electrical stimulation currents to form the mixed-frequency stimulation current, in which the electrical energies of the stimulation currents that have frequencies from 100 kHz to 200 kHz account for 30% to 100% of the electrical energy of the mixed-frequency stimulation current.

Moreover, another control method configured to control an electrical stimulation circuit of an ejaculation control nerve regulating device in the present invention comprises:
Outputting single frequency: turn on a power source and power up an electrical stimulation circuit; a mixed-frequency circuit of the electrical stimulation circuit controls a multi-frequency stimulation circuit to output an electrical stimulation current having single frequency to an outputting electrode;
Outputting mixed-frequency stimulation current: repeat outputting single frequency for multiple times, and the frequency of the electrical stimulation current of at least one of the multiple times is between 100 kHz and 200 kHz.

Further, the control method as mentioned above, wherein a duration of the stimulation current having frequency from 100 kHz to 200 kHz accounts for 30% to 100% of a duration of a continuous contact and conduction.

Two control methods of the electrical stimulation circuit for the ejaculation control nerve regulating device are provided in this disclosure, and said control methods include weighted frequency mixing control method and time-based frequency mixing control method, specifically illustrated as follows:

As shown in FIG. 5, the weighted frequency mixing control method includes:
Step of outputting multiple stimulation currents: turn on the power source 13 and power up the electrical stimulation circuit 12, and the multi-frequency stimulation circuit 121 of the electrical stimulation circuit generates and outputs multiple electrical stimulation currents C1 to the mixed-frequency circuit 122, and the electrical stimulation currents C1 have different frequencies respectively. The frequency range of said frequencies of the electrical stimulation currents C1 is between 10 kHz and 200 kHz, and the frequency of at least one of the electrical stimulation currents C1 ranges between 100 Hz and 200 kHz; and
Step of frequency-mixing and outputting: the mixed-frequency circuit 122 receives the multiple electrical stimulation currents C1 and outputs a mixed-frequency stimulation current C2 under a mixed-frequency mode to the outputting electrode 10.

The mixed-frequency mode is that the mixed-frequency circuit 122 simultaneously receives the multiple electrical stimulation currents C1, and mixes the electrical energies of the multiple electrical stimulation currents C1 to form the mixed-frequency stimulation current C2, in which the electrical energies of the electrical stimulation currents C1 that have frequencies from 100 kHz to 200 kHz account for 30% to 100% of the electrical energy of the mixed-frequency stimulation current C2, and preferably, the electrical energies of the electrical stimulation currents C1 that have frequencies from 100 kHz to 200 kHz account for 50% to 100% of the electrical energy of the mixed-frequency stimulation current C2.

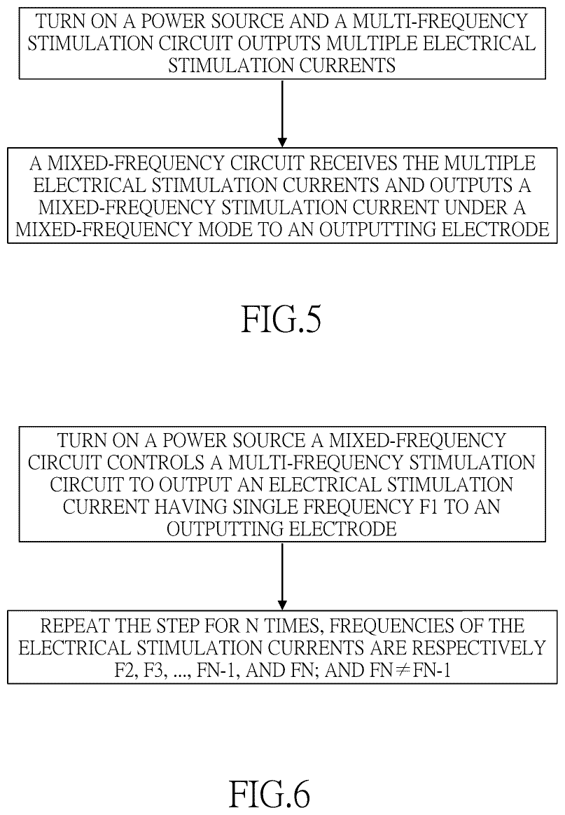

As shown in FIG. 6, the time-based frequency mixing control method includes:
Step of outputting single frequency: turn on the power source 13 to power up the electrical stimulation circuit 12, and the mixed-frequency circuit 122 of the electrical stimulation circuit 12 controls the multi-frequency stimulation circuit 121 to output an electrical stimulation current C1 having single frequency to the outputting electrode 10; and
Step of outputting mixed-frequency stimulation current: repeat the step of outputting single frequency for multiple times, and an interval t, in which t≥0, is between every two said times of step of outputting single frequency, and each time the frequency of the output electrical stimulation current C1 is different from the previous one, and the frequency range is between 10 kHz and 200 kHz; in which, the frequency of at least one of the output electrical stimulation currents C1 is between 100 kHz and 200 kHz.

Preferably, a duration of the electrical stimulation current C1 having frequency from 100 kHz to 200 kHz accounts for 30% to 100% of a duration of outputting the mixed-frequency stimulation current C2, and favorably, the duration of the electrical stimulation current C1 having frequency from 100 kHz to 200 kHz accounts for 50% to 100% of the duration of outputting the mixed-frequency stimulation current C2.

As shown in FIG. 4, when using the ejaculation control nerve regulating device in this disclosure, let the stimulating component 2 envelop the penis 3 and the stimulating electrode 22 contact a surface at a site corresponding to the dorsal nerves of the penis 3, and then let the outputting electrode 10 of the main unit 1 of the ejaculation control nerve regulating device contact the contacting electrode 21 of the stimulating component 2. Then turn on the power source 13 to power up the electrical stimulation circuit 12, and the main unit 1 would control the ejaculation control nerve regulating device to apply electrical stimulation to the dorsal nerves of penis via the weighted frequency mixing control method or the time-based frequency mixing control method.

Hereinafter, the technical solutions of the present invention will be further described with reference to specific embodiments.

### Embodiment A

A charging receptacle is disposed on a surface of the casing 11 in this embodiment, and the charging receptacle is electrically connected to the power source 13. The charging receptacle 15 is connected to an external power source via a cable to charge the power source 13.

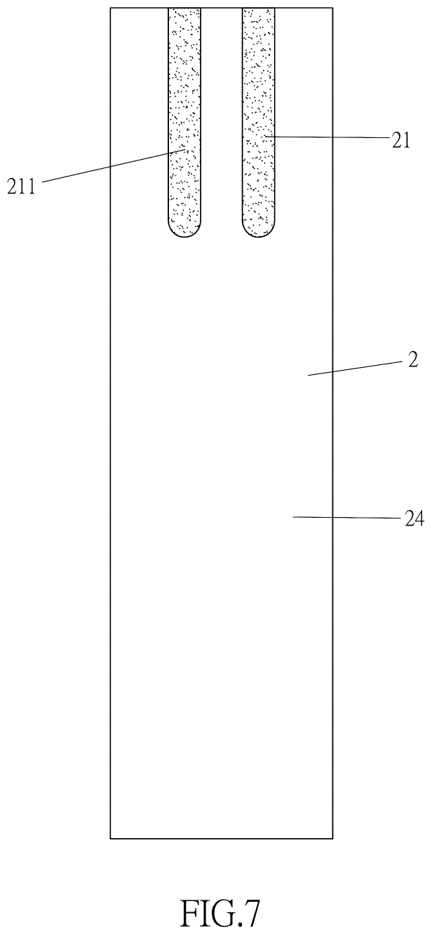

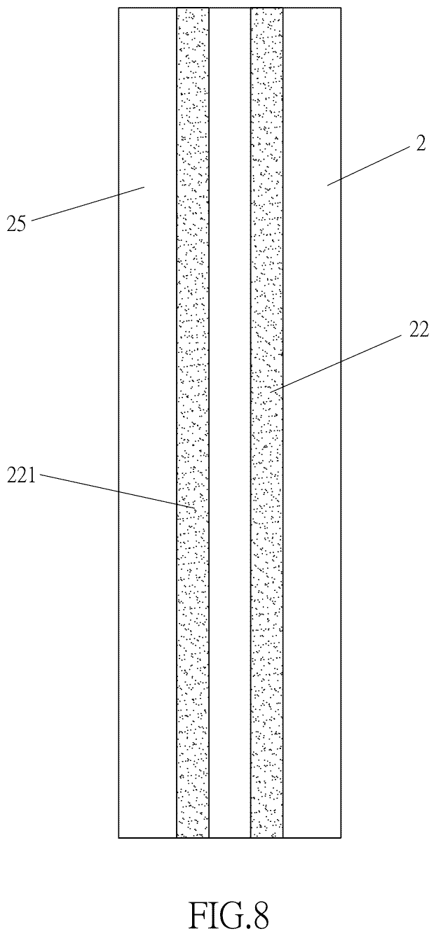

With reference to FIGS. 2, 7, and 8, the stimulating component 2 in this embodiment is a ring-shaped sleeve formed by end-to-end connection of a strap fabricated with an elastic material, but it is not limited thereto, the material of the stimulating component may be silicone materials being elastic, and the shape may be cup-shaped or ring-shaped. Each of two ends of the accommodating space 23 has an opening 26. The contacting electrode 21 is mounted on the outer surface 24 of the stimulating component 2, and the contacting electrode 21 includes at least two contacting points 211 being large-area metal pieces. The stimulating electrode 22 is mounted on the inner surface 25 of the stimulating component 2, and the stimulating electrode 22 includes at least two electrode contacting points 221 being strip-like and large-area; the length of said electrode contacting point 221 being large-area is at least a half of the circumference of an inner wall of the ring-shaped sleeve, thereby being capable of covering all of the nerves on the penis when the stimulating component 2 sleeves on the penis, ensuring the thoroughness and the accuracy of the treatment effect.

A gap distance between the electrode contacting points 221 is at least 1 mm and less than 30 mm to reach the effective electric field range, thereby being capable of effectively influencing nerves and conducting electrical stimulation treatment.

In this embodiment, the patient is treated with the time-based frequency mixing control method, in which the durations of the high-frequency stimulation currents having frequencies from 100 kHz to 200 kHz account for 50% of the duration of the output mixed-frequency stimulation current, and the durations of the low-frequency stimulation currents having frequencies from 10 kHz to 100 kHz account for 50% of the duration of the output mixed-frequency stimulation current.

The detailed parameters of frequency-mixing is that the mixed-frequency circuit controls the multi-frequency stimulation circuit to output a low-frequency stimulation current from 10 kHz to 20 kHz lasting for 50 milliseconds (ms) to 60 ms; after an interval of 450 ms to 500 ms, the mixed-frequency circuit controls the multi-frequency stimulation circuit to output a high-frequency stimulation current from 190 kHz to 200 kHz lasting for 50 ms to 60 ms; after another interval of 450 ms to 500 ms, the mixed-frequency circuit controls the multi-frequency stimulation circuit to output an electrical stimulation current from 10 kHz to 20 kHz lasting for 50 ms to 60 ms; and so the process repeats.

### Verification Experiment:

Twenty patients with refractory premature ejaculation, unresponsive to electrical stimulation treatment below 1 kHz, were selected as study subjects, and all of the subjects met the diagnostic criteria outlined in the International Society of Sexual Medicine Guidelines for premature ejaculation. Each one of the subjects has a regular female sexual partner and has had a stable heterosexual relationship for at least six months, having sexual intercourse at least twice per week and maintaining stability throughout the study period, and has withdrawn from other premature ejaculation treatments/products for more than three months. The intravaginal ejaculation latency time (IELT) of each one of the patients was from 15 seconds to 1 minute.

The twenty patients were treated with the Embodiment A of the ejaculation control nerve regulating device one minute per time and three times per day, and continued for one month. Data of the average IELT before and after the one-month treatment were respectively collected and as shown in Table-1.

TABLE 1
Comparisons of IELT before and after the treatment

| | IELT before treatment | IELT after one-month |
|---|---|---|
| | (seconds) | treatment (seconds) |
| patient 1 | 15 | 80 |
| patient 2 | 30 | 120 |
| patient 3 | 30 | 150 |
| patient 4 | 20 | 120 |
| patient 5 | 60 | 120 |
| patient 6 | 60 | 180 |
| patient 7 | 20 | 120 |
| patient 8 | 30 | 600 |
| patient 9 | 60 | 300 |
| patient 10 | 20 | 45 |
| patient 11 | 10 | 50 |
| patient 12 | 10 | 10 |
| patient 13 | 10 | 15 |
| patient 14 | 30 | 180 |
| patient 15 | 60 | 140 |
| patient 16 | 60 | 150 |
| patient 17 | 10 | 50 |
| patient 18 | 30 | 80 |
| patient 19 | 60 | 300 |
| patient 20 | 60 | 480 |

### Results:

During the experiment, none of the twenty patients reported any adverse reaction, showing good safety. In addition, all of the twenty patients were able to operate the device to complete the treatment at home by themselves, and none of the subjects withdrew from the study, which shows that the device is convenient to operate and leads to good adherence in use.

As shown in the Table-1, for all patients except patients 12 and 13 (underlined), the IELT was two-fold or higher than before the treatment, and 11 patients of which (shown bold in Table-1, over 50% of the subjects) had their IELT be five-fold or higher than before the treatment; in addition, an increasing trend in improving premature ejaculation was observed as the treatment time extended.

### Embodiment B

In this embodiment, the patients were treated with the time-based frequency mixing control method, in which the frequencies of the currents were the same as Embodiment A, and the proportions of low-frequency currents and the high-frequency currents accounted for the duration of the mixed-frequency stimulation current were also the same as Embodiment A. The specific parameters of the time-based frequency-mixing were: the low-frequency stimulation current continuously output for 10 ms to 20 ms, and immediately followed by switching to high-frequency stimulation current and output for 10 ms to 20 ms, and then stopped after so cycled for 100 ms; after an interval of 800 ms to 1000 ms, restarted with outputting low-frequency stimulation current, and so cycled.

### Verification Experiment:

Forty patients diagnosed with primary premature ejaculation were selected, withdrawing from all of the treatments for more than three months and having stable frequencies in sexual activities. The patients were randomly assigned to the experimental group (n=20) and the control group (n=20); the subjects in the experimental group were treated with Embodiment B of the ejaculation control nerve regulating device, and the subjects in the control group were treated with sensitivity-reducing devices that apply electrical stimulation currents with frequency from 200 kHz to 800 kHz. Other parameters of the electrical stimulation currents provided by the devices of the two groups were all identical. The patients in both groups were continuously treated for two weeks, twice per day, and one minute per time, and the data of the average IELT patients before and after the treatment of the forty patients were respectively collected after two weeks, in which the ejaculation latency time that extends more than one-fold was deemed effective. Observation in the safety was mainly screening on whether adverse reactions such as erectile dysfunction or rigidity decreasing were induced.

The data of the experimental group were shown in Table-2, and the data of control group were shown in Table-3.

TABLE 2
Results of the experimental group

| | Ejaculation latency | Ejaculation latency | Adverse |
|---|---|---|---|
| | time before | time after treatment | reaction after |
| | treatment (seconds) | (seconds) | treatment |
| patient 1 | 30 | 70 | None |
| patient 2 | 60 | 140 | None |
| patient 3 | 120 | 370 | None |
| patient 4 | 15 | 40 | None |
| patient 5 | 60 | 240 | None |
| patient 6 | 40 | 180 | None |
| patient 7 | 60 | 140 | None |
| patient 8 | 45 | 100 | None |
| patient 9 | 30 | 180 | None |
| patient 10 | 50 | 120 | None |
| patient 11 | 30 | 50 | None |
| patient 12 | 120 | 500 | Rigidity |
| | | | decreasing |
| patient 13 | 45 | 100 | None |
| patient 14 | 10 | 15 | None |
| patient 15 | 15 | 100 | None |
| patient 16 | 60 | 150 | None |
| patient 17 | 120 | 360 | None |
| patient 18 | 360 | 540 | None |
| patient 19 | 15 | 60 | None |
| patient 20 | 60 | 180 | None |

TABLE 3
Results of the control group

| | Ejaculation latency | Ejaculation latency | Adverse |
|---|---|---|---|
| | time before | time after treatment | reactions |
| | treatment (seconds) | (seconds) | recorded |
| patient 1 | 40 | 130 | None |
| patient 2 | 60 | 150 | None |
| patient 3 | 30 | | Erectile |
| | | | dysfunction |
| patient 4 | 40 | 60 | Rigidity |
| | | | decreasing |
| patient 5 | 10 | 30 | None |
| patient 6 | 20 | 50 | None |
| patient 7 | 60 | 180 | Rigidity |
| | | | decreasing |
| patient 8 | 40 | 120 | |
| patient 9 | 30 | 100 | |
| patient 10 | 15 | 40 | |
| patient 11 | 20 | 30 | |
| patient 12 | 30 | 110 | Rigidity |
| | | | decreasing |
| patient 13 | 40 | 120 | |
| patient 14 | 30 | | Erectile |
| | | | dysfunction |
| patient 15 | 45 | 100 | |
| patient 16 | 30 | 70 | |
| patient 17 | 30 | | Erectile |
| | | | dysfunction |
| patient 18 | 120 | 400 | |
| patient 19 | 100 | 240 | |
| patient 20 | 80 | 260 | |

### Results:

By comparing Table-2 and Table-3, it is observed that eighteen subjects were effective in the twenty patients of the experimental group, and the effective rate was 90%; only one patient experienced rigidity decreasing, and the rate of adverse reaction was 5%. Fifteen subjects were effective in the twenty patients of the control group, and the effective rate was 75%; three patients experienced erectile dysfunction and four patients experienced rigidity decreasing, and the adverse rate was 35%. Differences between the two groups in the effective rates and the adverse rates are statistically significant (P<0.05).

Conclusion: Embodiment B of the ejaculation control nerve regulating device has a high effective rate and meanwhile performs well in safety, being suitable for long-term use by patients with premature ejaculation to improve the sexual life quality.

This is primarily because the excitation of the dorsal nerves of penis could be inhibited by applying mixed-frequency electrical stimulation with high frequency (10 kHz to 200 kHz) but without completely blocking the nerve conduction, and thus decreases sensitivity partially on the penis but the patient still gets sensory reaction at the penis; maintaining sensory reaction can keep normal pleasure without affecting erection and rigidity. The electrical stimulation with frequency above 200 kHz, which provides strong effect in blocking nerves or may lead to heating effect or high temperature at the stimulating component, is easy to block normal nerve conduction about sexual signals when reducing sensitivity partially on the penis, and further affects normal penile functions like erection and rigidity, resulting in a high adverse reaction rate.

### Embodiment C

This embodiment is provided with the control method of weighted frequency-mixing, i.e., the multi-frequency stimulation circuit generates two electrical stimulation currents having different frequencies simultaneously and outputs to the mixed-frequency circuit, in which the low-frequency stimulation current having frequency ranging from 10 kHz to 100 kHz and the high-frequency stimulation current having frequency ranging from 100 kHz to 200 kHz are included.

The mixed-frequency circuit mixes the electrical energy generated by the low-frequency stimulation current and the electrical energy generated by the high-frequency stimulation current to form the mixed-frequency stimulation current and outputs to the outputting electrode. In which, the electrical energy of the low-frequency stimulation current accounts for 50% of the electrical energy of the mixed-frequency stimulation current, and the electrical energies of the high-frequency stimulation current accounts for 50% of the electrical energy of the mixed-frequency stimulation current.

In this embodiment, the low-frequency stimulation current has frequency ranging from 10 kHz to 20 kHz, and the high-frequency stimulation current has frequency ranging from 100 kHz to 200 kHz. Other designs are the same as Embodiment A.

With reference to the Embodiment A and Embodiment B, the benefits of the ejaculation control nerve regulating device in this disclosure are:
(1). The ejaculation control nerve regulating device in this disclosure is small and is provided with split-unit design, thus being easy to carry and meanwhile preventing the penis from experiencing great pressure during long-term treatment as treated with the integrated nerve regulating device.
(2). The main unit and the stimulating component of the ejaculation control nerve regulating device in this disclosure conducts the electrical stimulation by contacting, avoiding usage of wires, thus being convenient for storage and carrying, and preventing entangling of wires and cables of the power source when in use.
(3). The stimulating component of the ejaculation control nerve regulating device in this disclosure is made with an elastic material and is size-adjustable according to the penis of the patient, and the stimulating electrode being curved is capable of covering all of the nerves of the penis, ensuring the thoroughness and the accuracy of the treatment effect; the design of gap distance to the stimulating electrode is capable of reaching the effective electric field range, and thus is capable of effectively influencing the nerves.
(4). The ejaculation control nerve regulating device in this disclosure outputs the mixed-frequency stimulation current to treat premature ejaculation from multiple electrical stimulation currents having frequency ranging from 10 kHz to 200 kHz via the time-based frequency-mixing control method or the weighted frequency-mixing control method; with the mechanisms of enhancing the brain center controlling over ejaculation and lowering excitation of the dorsal nerves of penis, not only increases the effective rate of treating premature ejaculation with nerve regulation, but also reduces the risks to the patient of experiencing adverse reactions like erectile dysfunction and erectile rigidity decreasing, increasing safety.
(5). The ejaculation control nerve regulating device in this disclosure may treat electrical stimulation via the mixed-frequency stimulation current, and thus reduces treatment time, generally treating 30 to 60 seconds per time and being effective immediately after treatment.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An ejaculation control nerve regulating device comprising:
a main unit having:
an electrical stimulation circuit including a multi-frequency stimulation circuit and a mixed-frequency circuit; the multi-frequency stimulation circuit capable of generating electrical stimulation currents in various frequencies, and the mixed-frequency circuit controlling the electrical stimulation circuit to output a mixed-frequency stimulation current;
an outputting electrode mounted on and exposed on an external surface of the main unit and electrically connected to the electrical stimulation circuit, receiving the mixed-frequency stimulation current output from the electrical stimulation circuit;
a stimulating component having a contacting electrode and a stimulating electrode, and the contacting electrode electrically connected to the stimulating electrode; and
when in use, the outputting electrode of the main unit contacts the contacting electrode of the stimulating component, thereby conducting the electrical stimulation currents and/or the mixed-frequency stimulation current generated by the electrical stimulation circuit to the stimulating electrode.

2. The ejaculation control nerve regulating device as claimed in claim 1, wherein the multi-frequency stimulation circuit is at least capable of outputting a low-frequency stimulation current and a high-frequency stimulation current, and a frequency of the high-frequency stimulation current is higher than a frequency of the low-frequency stimulation current.

3. The ejaculation control nerve regulating device as claimed in claim 2, wherein a frequency range of the high-frequency stimulation current is between 100 kHz and 200 kHz.

4. The ejaculation control nerve regulating device as claimed in claim 1, wherein, the stimulating component is configured to envelope a stimulated site; when the stimulating component envelopes the stimulated site, the contacting electrode is mounted on an outer side of the stimulating component, and the stimulating electrode is mounted on an inner side of the stimulating component and is capable of attaching to the stimulated site.

5. The ejaculation control nerve regulating device as claimed in claim 3, wherein, the stimulating component is configured to envelope a stimulated site; when the stimulating component envelopes a stimulated site, the contacting electrode is mounted on an outer side of the stimulating component, and the stimulating electrode is mounted on an inner side of the stimulating component and is capable of attaching to the stimulated site.

6. The ejaculation control nerve regulating device as claimed in claim 4, wherein the stimulating component is made of an elastic material, thereby being capable of covering the stimulated site in variable sizes.

7. The ejaculation control nerve regulating device as claimed in claim 5, wherein the stimulating component is made of an elastic material, thereby being capable of covering the stimulated site in variable sizes.

8. The ejaculation control nerve regulating device as claimed in claim 6, wherein the stimulating electrode includes at least two contacting points, and a gap distance between the at least two contacting points is between 1 millimeter (mm) and 30 mm.

9. The ejaculation control nerve regulating device as claimed in claim 7, wherein the stimulating electrode includes at least two contacting points, and a gap distance between the at least two contacting points is between 1 mm and 30 mm.
